(19) United States
(12) Patent Application Publication
(10) Pub. No.: US 2016/0143765 A1
(43) Pub. Date: May 26, 2016

(54) Compression Garment for Postpartum Women

(71) Applicant: Shajine Blake, Houston, TX (US)

(72) Inventor: Shajine Blake, Houston, TX (US)

(21) Appl. No.: 14/553,903

(22) Filed: Nov. 25, 2014

(51) Int. Cl. A61F 5/02

(52) U.S. Cl. CPC A61F 5/02

(57) ABSTRACT

A postpartum compression garment for covering and the application of pressure to the lower torso that includes a back panel and at least three elastic straps originating at the sides of back panel that are sized to extend from a first lateral side to a second opposite lateral side of the back panel to cover the Transverse Abdominis muscles wherein the straps can be releasably attached to provide different tension and overlap one another.

### FIELD OF THE INVENTION

The present invention relates to the field of garments and other apparel and particularly to compression garments for postpartum use.

### BACKGROUND OF THE INVENTION

Compression garments are made from a stretchable material that tightly conforms to the body. The degree of compression provided by a given conventional compression garment is dependent on the wearer's individual body shape and size and the garment. Compression garments are used by postpartum women to support the back and abdominal muscles after pregnancy, offering the new mother comfort. As the hormones reset, ligaments can still be loose and a compression garment may add some stability while new moms are lifting and bending as well as sculpt the body. It has been further reported that compression garment can support uniform skin shrinkage, an increase in venous and lymphatic drainage. After giving birth, the uterus is still enlarged and the abdominal wall has lengthened and stretched throughout the pregnancy. A wrap across the stomach area after delivery is reported to help shrink the uterus, retract the muscles of the abdominal wall and reduce the space for fat deposits.

Different people wearing the same compression garment may have significantly different waist sizes which results in different amounts of compression provided by the garment. In addition to implicit differences based upon the respective sizes of the users, each user may have personal preferences with respect to the level of compression sought as well as the locations. While exercises like crunches and oblique curls assist with muscle tone and can help tone muscle after pregnancy, these conventional exercises generally work the external layers of the abdominal wall. After pregnancy, the external abdominal muscles easily overpower the relatively weaker and deeper abdominal muscles in this region, the Transverse Abdominis. The Transverse Abdominis when contracted compresses and flattens the abdomen. Conventional exercises that fail to target the Transverse Abdominis can cause the belly to balloon outward. Moreover, the bulging of the abdominal wall can create additional problems like abdominal separation and low back pain and worse may prevent the flattening of the abdomen. Therefore, one recommended manner in which to flatten the abdominal muscles after pregnancy is to target the Transverse Abdominis muscles with exercise.

While there a number of garment products that have been disclosed that allow a user to adjust compression in desired locations, the present invention specifically address the unique needs of postpartum women and provides an adjustable garment that can provide both a targeted and adjustable compression on the area of a women's torso spanning the pelvic area to an area below the breast area and divides this region into at least three zones.

Therefore, it is an object of the invention to provide an adjustable compression garment for the torso that allows a user to adjust the compression of the torso region in at least three zones that generally cover the same area as the Transverse Abdominis muscles and which extend from a user's pelvic area to a user's mid torso.

It is further object of the invention to provide a compression garment that can assist with targeting the Transverse Abdominal muscles during exercise and mitigate the bulging of the abdominal wall.

### SUMMARY OF THE INVENTION

The present invention is directed to an adjustable and targeted compression garment and comprises a plurality of straps that extend from lateral side of a shirt that are moveable relative to the shirt and positioned to cover the the Transverse Abdominis muscles. The straps are made from a stretchable fabric material that, when fastened, can provide a varying degree of compression to targeted areas of the torso.

A fastening arrangement between the distal loose end of the strap and the garment includes a first fastener device connected to a fabric portion of the strap and a second fastening device that is connected to the shirt. The fastening device provides an adjustable connection between the first fabric portion of the strap and the second fabric portion of the garment. In an embodiment the fastener uses a hook and loop arrangement such as that sold under the brand name Velcro.

In alternative embodiments, the straps are fastened using a buckle or threaded c-clips. In yet a further embodiments, the fastening arrangement comprises a unidirectional hook and loop arrangement, where the hooks and loops are configured to allow the first fabric to slide over the second fabric without releasing the first fabric from the second fabric. In each of the embodiments, the degree of compression may be adjusted by the user. In some of the embodiments, the adjustment can be achieved without completely releasing the strap from its point of attachment which thereby allows the user to more easily cinch the strap and progressively tighten the connection.

In at least one embodiment, the adjustable compression garment includes an elastic material that is provided on both the torso section of the garment and on the straps. Other embodiments use different combination of fabric with different degrees of elasticity some of which substantially lack elasticity. The garment is particularly suitable for postpartum use.

The above described features and advantages, as well as others, will become more readily apparent to those of ordinary skill in the art by reference to the following detailed description and accompanying drawings.

### BRIEF DESCRIPTION OF THE DRAWINGS

FIG. 1 is a front view of a garment according to a first embodiment of the invention.

FIG. 2 is a front view of the garment depicted with the compression straps disengaged.

FIG. 3 is a lateral view of the embodiment depicted in FIG. 1.

FIG. 4 is the opposite lateral view of the embodiment depicted in FIG. 1.

FIG. 5 is front view of an alternative embodiment of the invention.

FIG. 6 is a rear view of yet a further embodiment of the invention.

FIG. 7 is a front view of the embodiment of FIG. 6.

FIG. 8 is a lateral side view of the embodiment of FIG. 6.

FIG. 9 is a perspective view of an alternative fastening hardware that may be used with the invention.

FIG. 10 is a perspective view of an alternative fastening hardware that may be used with the invention.

FIG. 11 is a perspective view of an alternative fastening hardware that may be used with the invention.

FIG. 12A is a perspective view of an alternative fastening hardware that may be used with the invention.

FIG. 12b is another perspective view of an alternative fastening hardware used with the invention.

FIG. 13 is a perspective rear view of yet a further embodiment of hardware that may be used with embodiments the invention.

FIG. 14 is a front view of a further embodiment of the invention with the straps unsecured.

FIG. 15 is a front view of the embodiment of FIG. 14 with the straps in a secured position.

FIG. 16 is a side view of the embodiment depicted in FIG. 14 with the straps secured.

FIG. 17 is the opposite a side view of the embodiment depicted in FIGS. 14-16.

FIG. 18 is a side view in elevation of a further embodiment of the invention.

FIG. 19 is a front view in elevation of a further embodiment of the invention.

FIG. 20 is a side view in elevation of the embodiment depicted in FIGS. 18 and 19.

### DETAILED DESCRIPTION

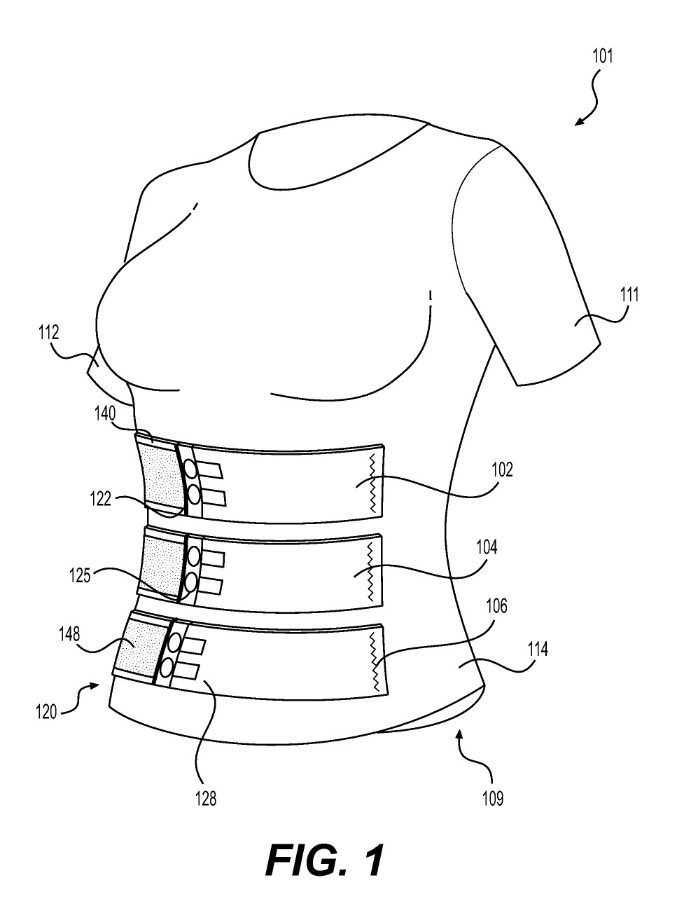

Now referring to FIG. 1, a first embodiment of a garment with adjustable compression is shown in the form of a compression shirt 101 with three elongate straps 102, 104, and 106 that originate from and are affixed to the lateral side 114 of the garment 101. In the embodiment depicted in FIG. 1 the bottom of the garment has an opening 109 that allows the user to easily pull the garment over the head. The garment has sleeves 11 and 112 to receive the wearer's arms. In the embodiment depicted in FIG. 1, straps 101, 104 and 106 are sewn to the garment along the later side 114 of the garment and are designed to span the front of the torso and lie relatively flat. The opposite ends 122, 125 and 128 of straps 101 104 ad 106 are depicted in an engaged position with opposite engagement elements 149, 145 and 148. Engagement elements 140, 145 and 148 are attached to the garment on the opposite side of the garment and are also sewn in place.

Shirt 101 and straps 102, 104 and 106 are comprised of an elastic compression material that made of a compression material such as a fabric including elastane (Spandex), polyester, nylon, neoprene and mixtures thereof, or other stretch fabrics (which may also be referred to herein as “elastic” materials or fabrics). Other fabrics having some modulus of elasticity may be used in the various embodiments of the garment and parts thereof and exhibit the adjustable compression techniques that are disclosed herein.

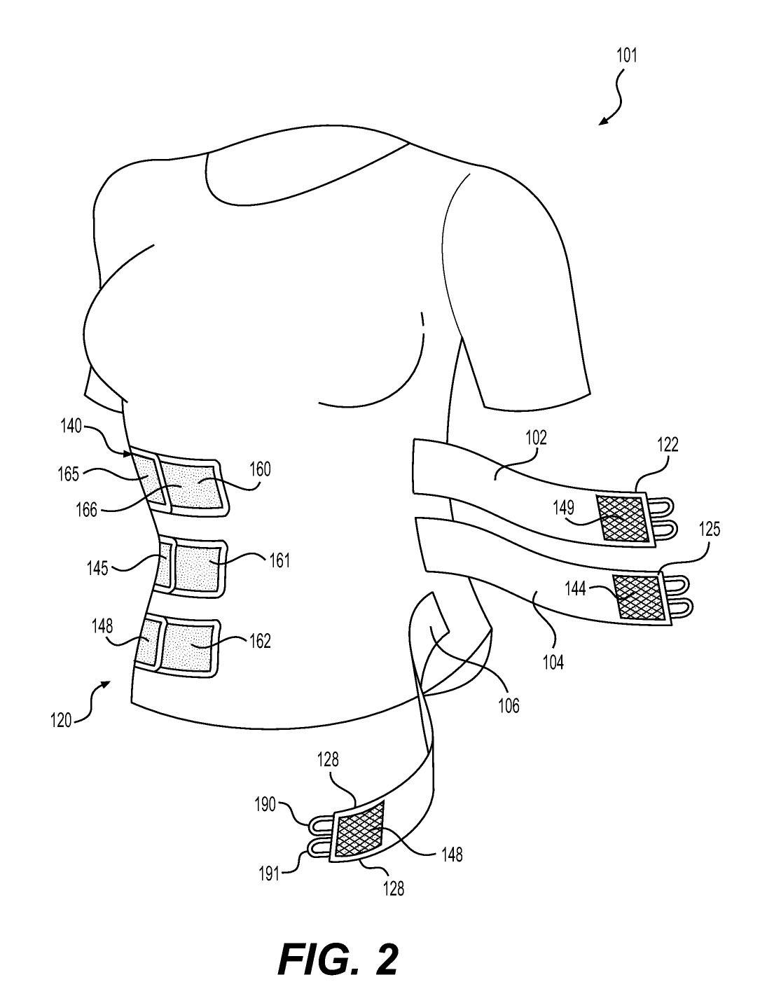

Referring now to FIG. 2 straps or 102, 104 and 106 are depicted with the distal ends free from engagement. The engagement member 140 145 and 148 are depicted that include short sections 160, 161 and 162 that have one end that is attached to the garment and an opposite end free and that extends from the lateral side 120. In this embodiment, the elements 140 comprise a loop elements 165 and 166 that are permanently secured to the engagement element which is, in turn, permanently secured to the garment 101. These loop elements 160, 161 and 162 engage opposite hook elements 140, 144 and 148. In the embodiment the two part engagement element provides a secure hold that can easily be adjusted. The ends of the straps include loops such as loops 190 and 191 that allow the user to easily grasp the straps, stretch the straps and manipulate the engagement element into place. Loops 190 and 191 are made of thermoplastic resin such as nylon, or high density polyethylene. Still referring to FIG. 2, straps 102 104 and 106 are provide with a hook engagement that is designed to firmly engage the opposite loops. In yet a further embodiment one side of the hook and loop fastener is an elongate section and is directly attached to the garment that allows the opposite side of the fastener that is located on the strap to be fastened at a variety of locations along its length.

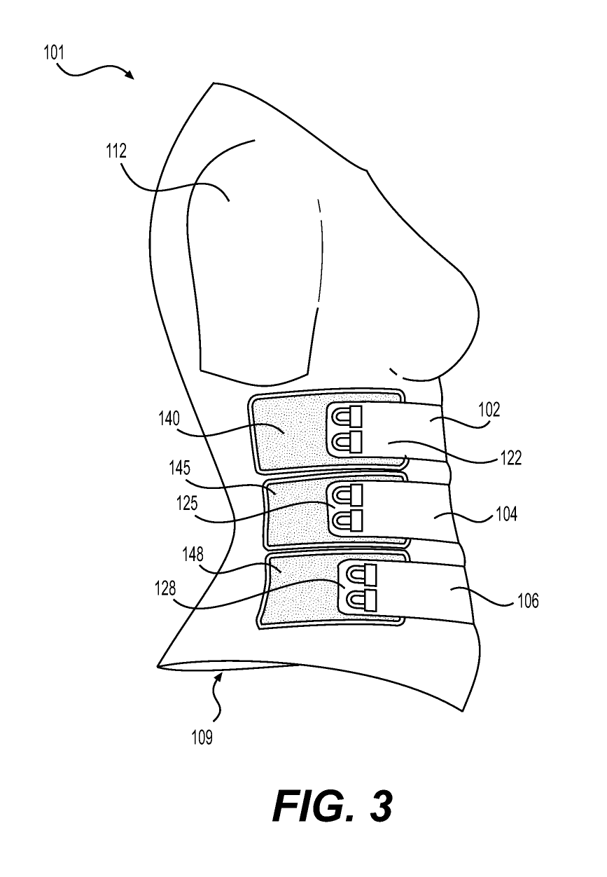

FIG. 3 is a side view of the embodiment depicted in FIG. 1. Which shows that the engagement surfaces 140, 145 and 146 extend from the front to the rear side of the garment and therefore allow the straps to be attached at the sides at one of a plurality of locations based upon the user's preference.

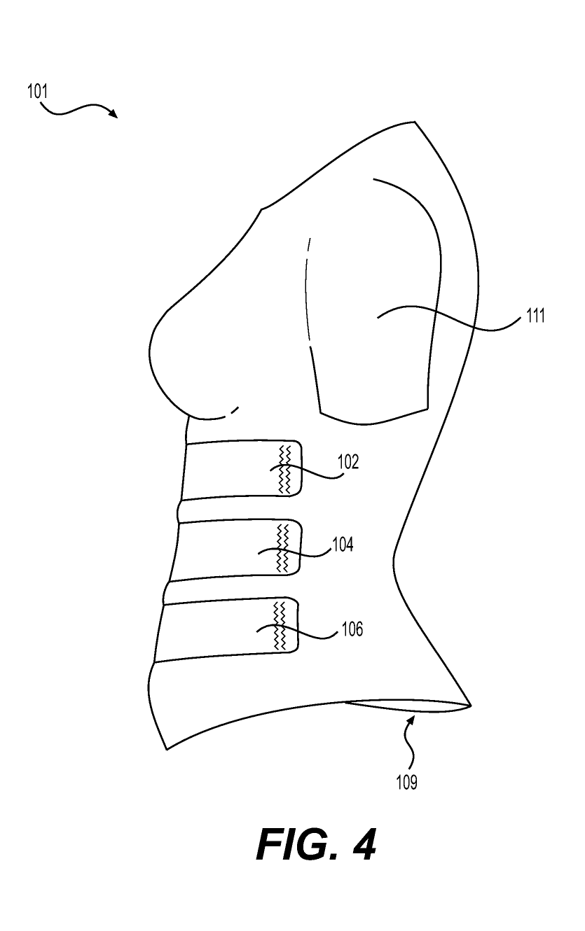

FIG. 4 depicts the right side of the garment depicted in FIG. 1 and shows the permanent attachment of the ends of the straps 102, 104 and 106 on the lateral side of the garment 101.

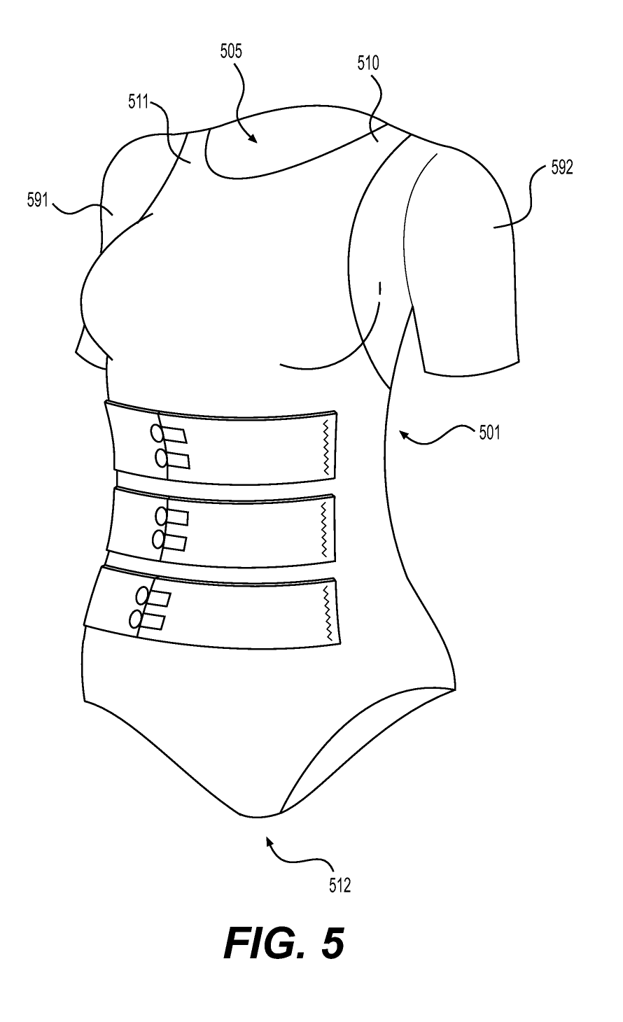

FIG. 5 depicts an alternative embodiment of a garment 501 that includes an integral panty portion 512. In this embodiment, the structure of the garment maintains the garment in position and the will tend to refrain from rising up past the pelvic area. The panty portion of the garment 512 may be continuous or be provided with a releasable closure that allows sanitary access. The garment depicted in FIG. 5 includes wide opening 505 that is defined by straps 510 and 511 that allow the garment to be accessed from the top. The straps also define openings for the wearer's arms 591 and 592. In alternative embodiments, access at is provided by an overlapping fabric sections such as overlapping fabric arrangements that are conventionally provided in male briefs.

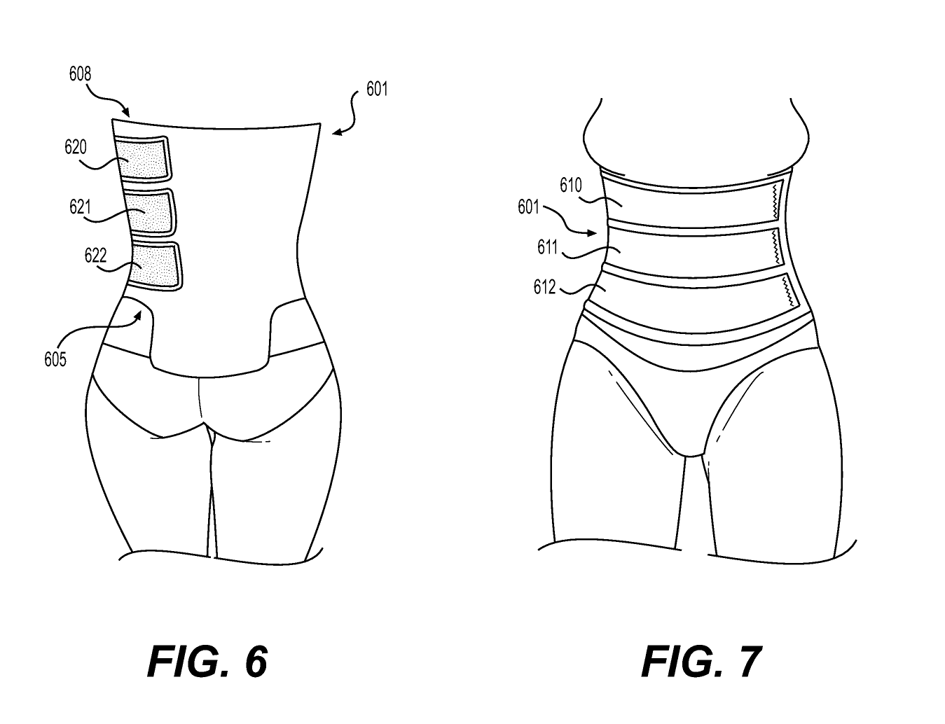

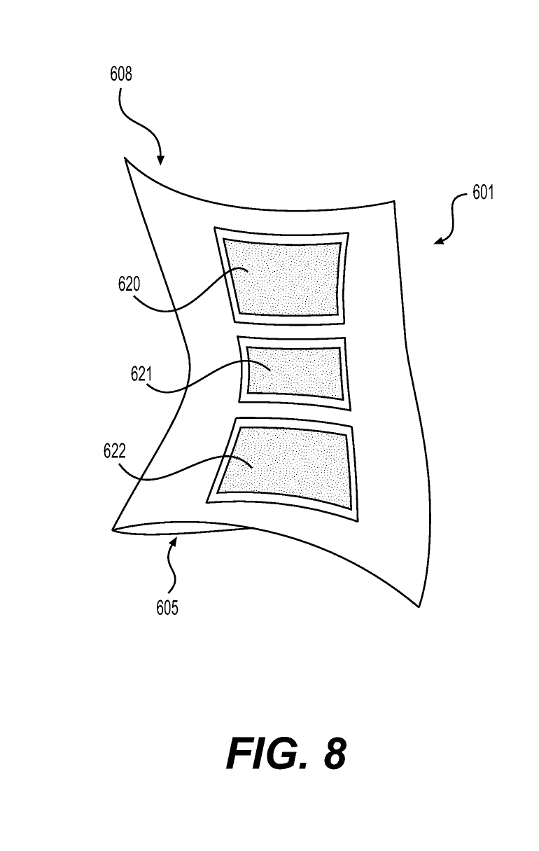

Now referring to FIGS. 6-8 an alternative embodiment garment 601 designed for immediate postpartum wear has an open bottom 605 and open top 608 and therefore comprises a body sleeve or corset. Garment 601 is designed to be worn below the bust. Garment 601 is easy to put on and allows a new mother to take advantage of a compression garment shortly after delivery and allows a new mother to use the garment to narrow her hips post delivery. Because each of the straps 611, 612 and 613 may be moved and operated separately, the individual can make any adjustments as needed or desired. Like the embodiment depicted in FIG. 1, the straps engage pads provided on the lateral sides of the garment that provide for releasable engagement. Since the garment includes separate bands, the user may open and release one band without affecting the other bands and compressed areas. This feature may assist with skin-to-skin bonding with the infant during breastfeeding and early postpartum interaction.

The ideal period for a woman to wear the garments according to the invention is 6-8 weeks postpartum. However, the longer the garment is worn the better the results will be provided the wearer follows and appropriate exercise and diet regimen. It is further contemplated that the garment may be continuous used as a daily wearer or as a supplemental garment to a user's exercise routine. As discussed above, the garment is designed to provide shape and structure of the core. The tightness of the band will restrict the muscles and trains them to remain in place and tighten as the wearer moves and exercises. An additional benefit to the garment is that it can provide a consistent restriction while eating and therefore mitigates the belly from expanding. During eating, the muscles of the stomach will expand and wearing the garment will maintain the muscles in place and will not allow the muscles to loosely and rapidly respond to the expansion of the stomach due to ingestion of food and fluids. As discussed above, the garment will also help retain tightness of the core and assist with the overall reduction in inches of the waist.

The embodiment depicted in FIG. 5 that includes an integral panty disclosed is also effective because it allows for the ease of daily wear under garments from pants to skirts, and prevents the garment from riding upwards, especially for active wear use such as running or during the performance of cardio exercise activities.

Each of the embodiments of the present invention also provides a stable structure to support breastfeeding moms who often experience back pains post labor and delivery due to changes in the body, breastfeeding and improper posture. As disclosed herein, the bands will substantially cover the entire abdominal area—the upper portion, the mid portion and lower portion. The preferred embodiment uses three bands, an upper strap that is designed to cover the upper torso and that is typically provided with a light compression, a center strap that can provide a mid-level of compression and a lower strap that may typically adjusted in a higher degree of compression that the other straps.

While the entire torso region is affected during pregnancy, the three zones and corresponding three straps allow for targeted and adjustable compression and assist with regaining muscle strength. While preferred embodiments use three bands, it is contemplated that four or more bands may also be advantageously used with the invention.

In view of the dramatic changes to the body caused by pregnancy, women often find it difficult to regain their pregnancy shape especially with respect to the lower abdominal region. The overlapping feature of the straps creates an increased compression to maximize the effect of skin tightening and the restructuring of the abdomen. The zones of the garment can assist with the creation of an hour glass shape for the wearer as each strap can be adjusted to varying degrees of tightness. In contrast, many of the prior art devices have a single strap or a single compression area that are all in one piece create a cylinder shape of the torso. The target compression area provided by the invention is divided into three regions that generally cover the abdominal region that has been stretched by the expansion of the uterus.

The compressing effect of the garment works to decrease swelling and inflammation postpartum. The length of the garment, which may extend to the hips, prevents riding and movement so the garment sits better on the desired area of the torso. The open bottom of the garment as disclosed in in some of the embodiments enhances both sanitary and comfort purposes and allows for the changing of sanitary napkins.

As discussed above, in alternative embodiments, the shirt may be comprised of a stretchable compression material such as a fabric including elastane, polyester, nylon, mixtures thereof, or other stretch fabrics (which may also be referred to herein as “elastic” materials or fabrics). However, other fabrics having some modulus of elasticity may be used in the various embodiments of the garment and exhibit the adjustable compression techniques that are disclosed herein.

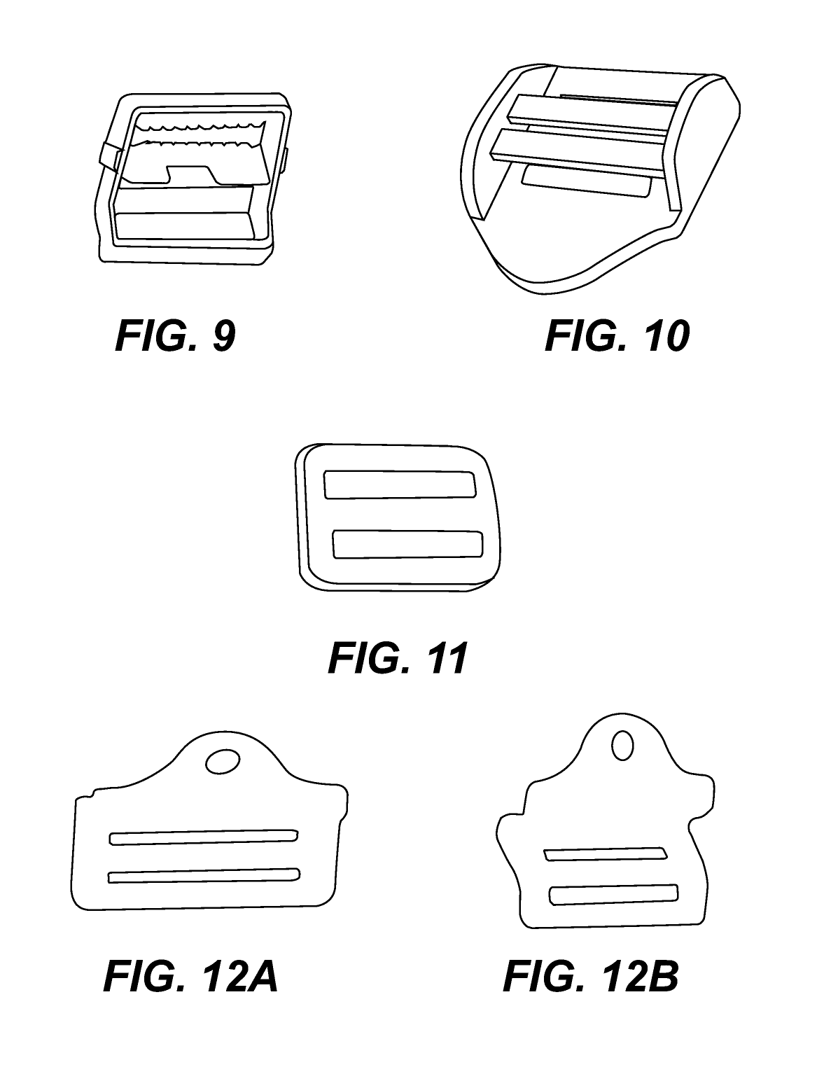

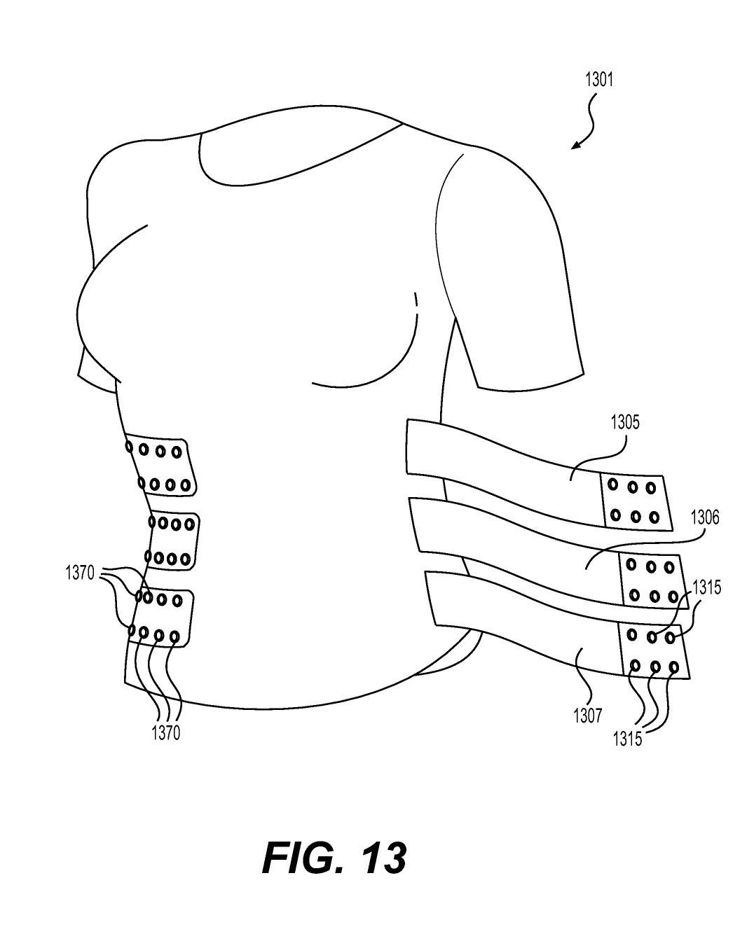

In addition to hook and loop fasteners as disclosed above, alternatively, the straps may be attached using a tri-glide or strap glide hardware such as that depicted in FIGS. 11 and 12a and 12b, ladder locks such as depicted in FIG. 9, or cinch locks as depicted in FIG. 10. In further embodiments the straps may also be provided with hook and eye type fasteners or snap fasteners. Now referring to FIG. 13, a further embodiment of the invention also uses three straps 1305, 1306 and 1307 and uses a plurality of points of attachment consisting of snap heads 1315 that can be engaged with snap bodies 1320 that are provided on the opposite side of the garment. The user can selected the head and body to provide for varying degrees of compression for each strap.

In yet a further an alternative embodiment, the compression adjustment arrangement is provided by a unidirectional hook and loop fastener arrangement, that that allows each of the straps to be adjusted on the garment in a variety of different positions. Each of the plurality of different positions stretches the fabric of the strap to a different degree such that a different degree of compression is provided to the torso of the individual wearing the shirt at each of the different positions.

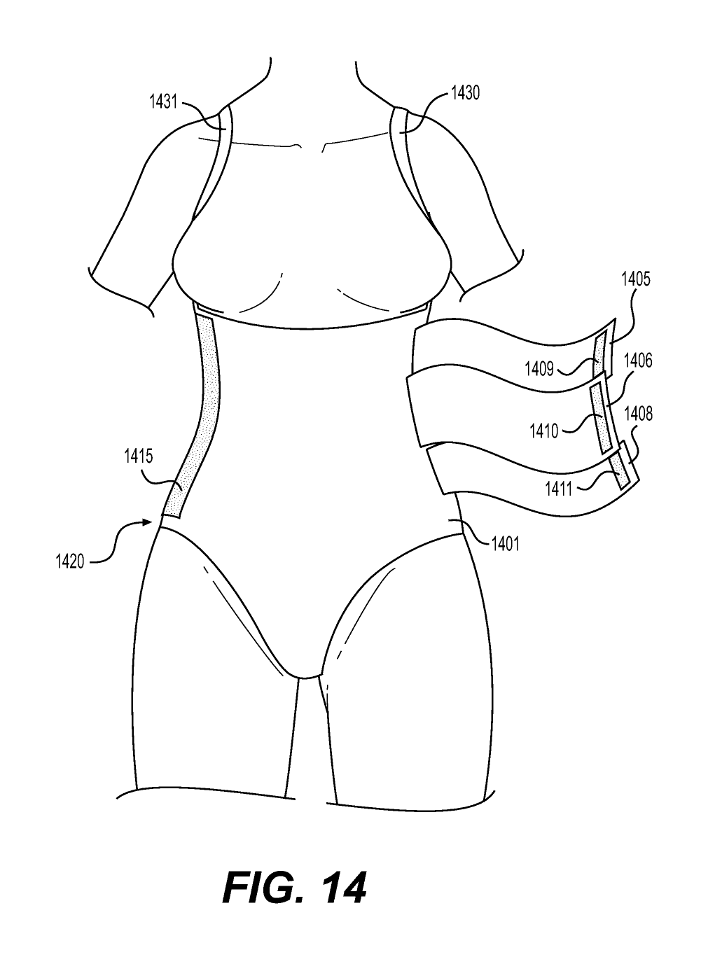

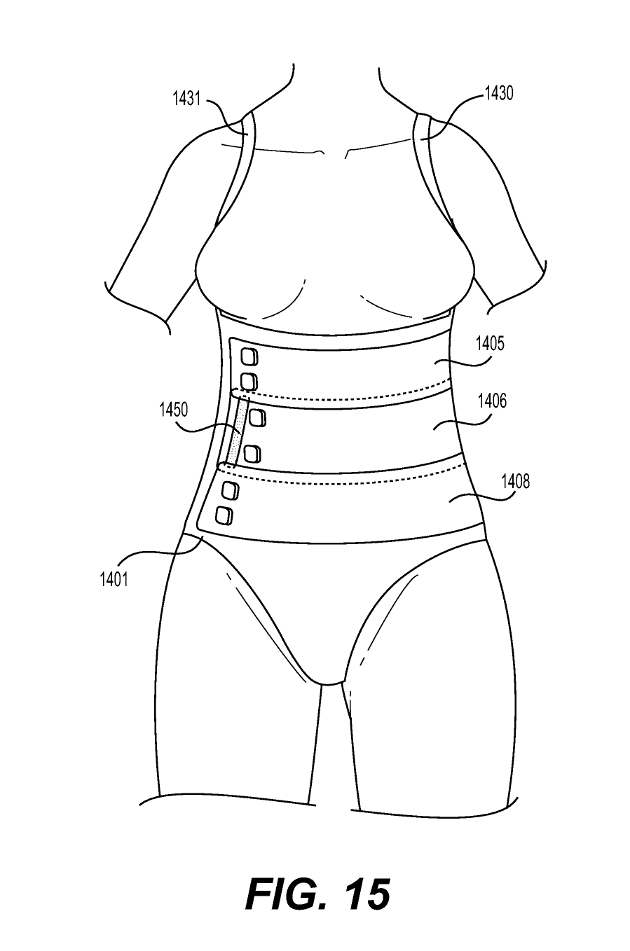

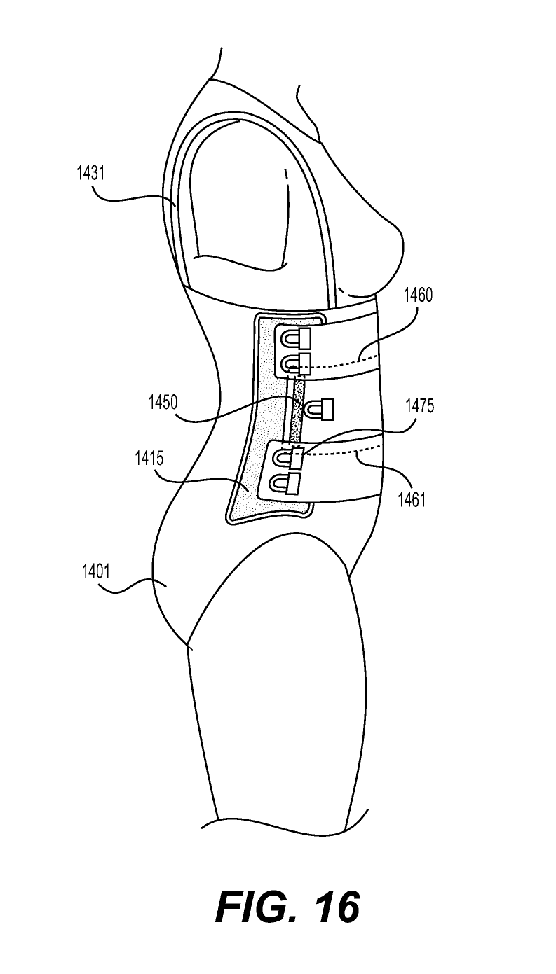

Now referring to FIG. 14, in a further embodiment the compression garment includes a plurality of straps that over-lap one another. In this embodiment the garment 1401 has three straps 1405, 1406 and 1408. The ends of the straps are provide with engagement regions 1409, 1410 and 1411 that consists of one side of a hook and loop type fastener such as Velcro. The garment 1401 has an opposite region 1415 that will receive and secure the ends of the straps. This embodiment covers the lower hip region 1420 and has suspenders 1430 and 1431 to assist keeping the garment in position. As best seen in FIG. 15, the center strap 1406 also has an engagement region 1450 on the exterior surface of the band that can received the opposite hook and loop type fasteners on the adjacent over lapping bands 1408 and 1405. The top and bottom edges 1460 and 1461 of middle band 1406 are covered by the adjacent bands and the engagement region 1450 extends beneath the respective adjacent bands. In embodiments each of the bans may be provided with such an engagement region to allow for a variety of overlapping configurations. For example, the top band 1405 may be crossed with the bottom band 1408 and each band 1405 and 1408 overlaps center bans 1406. Now referring to FIG. 16, a side view of the garment 1406 is depicted wherein the top and lower bands overlap the center band. The side of garment 1401 includes an extended hook and loop engagement region 1415 that provides for engagement at different tensions for the band as well as adjustments up and down its vertical length. Like other embodiments, the straps are provide with handles such as handle 1475 to allows the user to easily stretch the bands across the torso and into contact with the engagement region 1415.

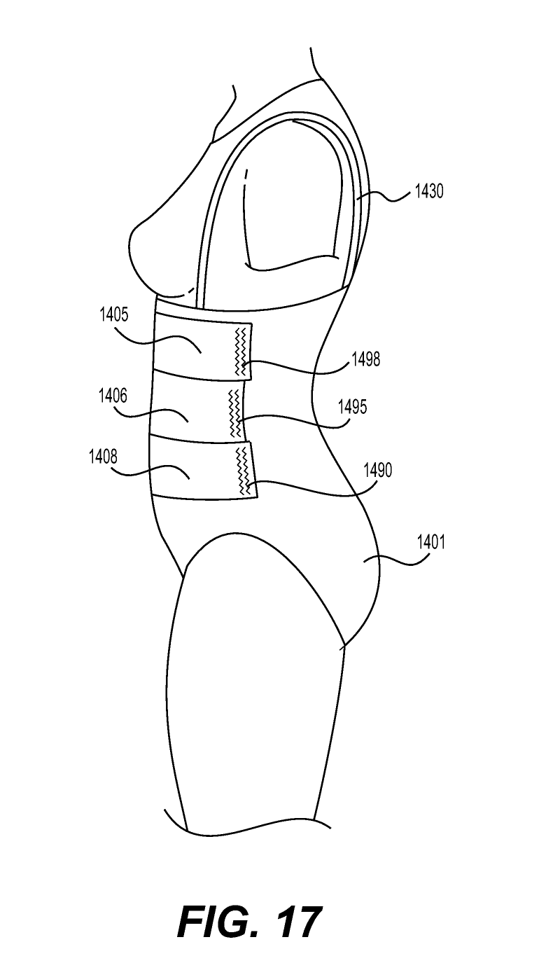

FIG. 17 depicts the opposite side view of garment 1401. As seen in FIG. 17, the three bands 1405, 1406 and 1408 are affixed to the garment to allow the bands to open and close across the front of the garment. In the embodiment the bands are sewn to the garment at the seam 1490, 1495 and 1498.

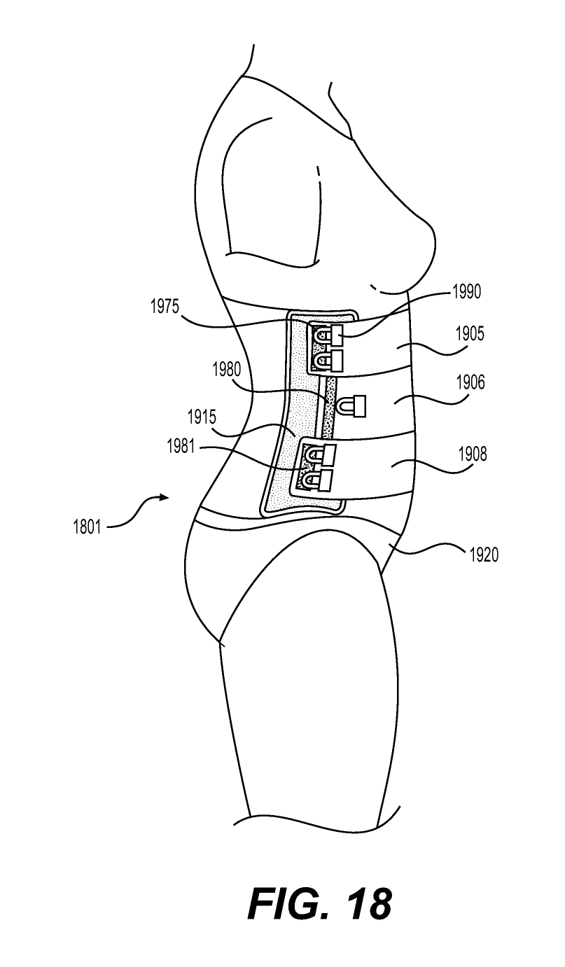

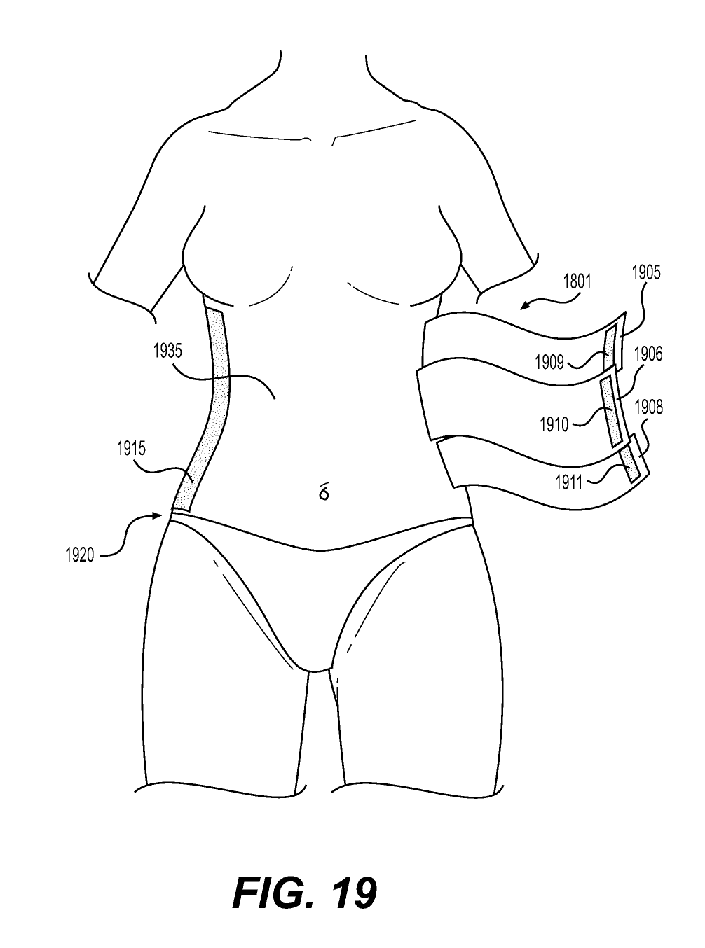

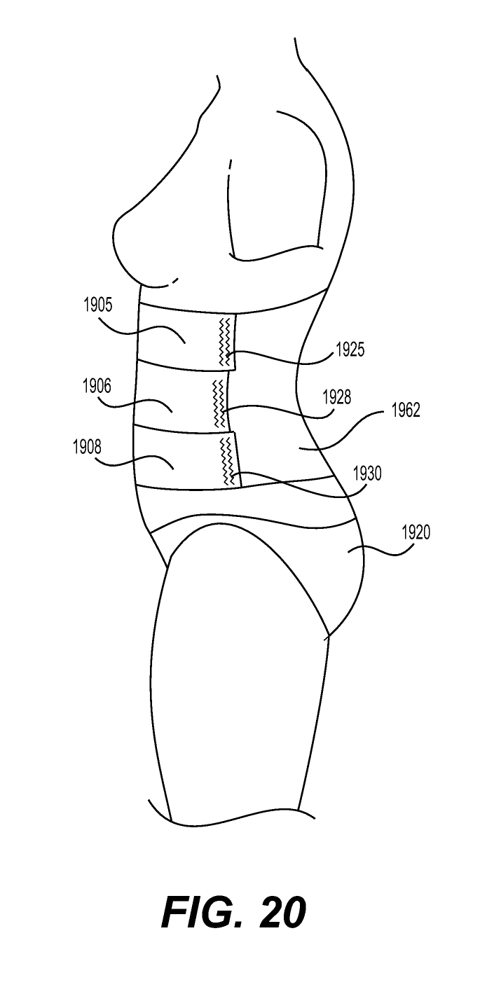

Now referring to FIG. 18 in a further embodiment of the invention, the garment 1801 is not tubular shaped but rather substantially comprises a plane when in an open position. Straps 1905 1906 and 1908 extend from a first lateral side to engage opposite fastener 1915 that provided on the opposite side of the garment. Straps 1905, 1906 and 1908 include fastener elements 1975, 1980 and 1981 on the external surface as well as fasteners on the inside surface (not shown). The presence of the external fastening surface allows the user to fasten the straps in an overlapping manner as well as on top of one another. In addition, the straps can cross one another in alternative engagement orientations. Loop members such as member 1990 are provide to manipulate the strap. As seen in FIG. 10, garment 1801 includes the straps 1905, 1906 and 1908 that cover the torso 1935 of the wearer. The inside surface of the straps include an engagement surfaces 1909, 1910 and 1911. This design, similar to a corset, does not include a built-in panty but rather is designed to be worn with a separate panty 1920. As shown in FIG. 20 the straps are attached by stitching 1925, 1928 and 1930 to rear panel 1961.

Advantages of this embodiment is that it is easy to put on, adjust and completely remove, which is especially advantageous after childbirth. Further, the straps can easily be unfastened to allow skin to skin contact between a newborn infant and the mother.

In the embodiments depicted in FIGS. 1-5 the shirt therefore generally comprises a torso portion, arm openings and an opening for the head and neck. The torso portion in each of the embodiments covers the torso and the straps are designed to provide increased compression to the regions generally defined by the transvers abdominal muscles. This area includes the hips and waist and ends just below the breasts. When the straps are stretched and the engagement pads is put in contact with the opposite engagement elements, the compression fabric is stretched to some degree around the torso of the wearer. Therefore, the straps provide an adjustable compression arrangement capable of a targeted and varying a degree of compression provided to the torso at the particular location based on the engagement positions which substantially cover the abdomen.

A lesser degree of compression may also be provided by the garment itself which, in embodiments, comprises an elastic compression document such as spandex. The materials used to provide the engagement elements on the ends of the straps may be any of a number of different fastener materials.

As discussed above, in one embodiment an engagement element provides hooks for a unidirectional hook and loop fastener arrangement. In these unidirectional hook and loop fastener arrangements, all of the hooks are oriented in the same direction instead of multiple different orientations as is typical with most hook and loop fasteners. Openings for receiving loops are formed between the tip of each hook and the base portion of the loop elements. Even though all of the hooks are oriented in the same direction with the unidirectional hook and loop arrangement, all of the loops are generally not oriented in the same direction. With such a unidirectional hook and loop arrangement, when the strap is engaged with the engagement loop element, the elements can slide across each other in a first direction but are prevented from movement when slid in the opposite direction. Because the ends of the hooks wrap around the loops, the engagement elements do not easily pull away from one another.

Molded unidirectional hook arrangements are advantageous because it does not easily snag fabric in one direction of movement. Furthermore, particles are also not easily collected in the hooks since there is no double-sided hook to lock particles in the arrangement. The term “unidirectional hook arrangement” refers to an arrangement where substantially all of the hooks of the fastener member are oriented in the same general direction. A further advantage to unidirectional hook and some of the buckle arrangements disclosed herein is that the compression adjustment can be progressively achieved by sliding the components relative to one another along a single planar and without the need to lift or separate the component away from the other or disconnect the two components during the process. Also, the compression adjustment arrangement will retain the compression fabric at a first degree of stretch, increase the degree of stretch strap that is engaged from the first degree of stretch to a second degree of stretch without releasing. In yet a further contemplated embodiment, the fastener uses a structure that is found on releasable zip ties including a sleeve and opposite saw tooth surfaces that can interlock and restrict rectilinear movement with respect to the opposite ends of the fastener elements.

In yet further embodiments, portions of the garment and portions of the straps may be comprised of fabric that is elastic and other portions that are relatively inelastic. For example, in an alternative embodiment the garment may have a rear panel that spans the lateral sides that is made from an inelastic fabric such as a synthetic leather material (or other synthetic material) and the front of the garment may be an elastic fabric. In yet further embodiments portions of the garment may be comprised of mesh sections may be provided to provide additional comfort and ventilation for the wearer. An advantage of the invention as described is that is allows for the selective compression of the region in which the straps cover.

The three band design allows the opening of one of the bands without affecting the other compressed areas. A further advantage of the invention is that it can accommodates skin-to-skin bonding during breastfeeding or early postpartum interaction with the infant. In addition, the back support feature provides a stable structure to support breastfeeding moms who often experience back pains post labor and delivery due to constant breastfeeding and improper posture

Although the present invention has been described with respect to certain preferred embodiments, it will be appreciated by those of skill in the art that other implementations and adaptations are possible. Moreover, there are advantages to individual advancements described herein that may be obtained without incorporating other aspects described above. Therefore, the spirit and scope of the appended claims should not be limited to the description of the preferred embodiments contained herein.

What is claimed is:

1. A postpartum compression garment for covering and the application of pressure on the lower torso comprising a back panel and at least three straps originating at a first lateral side of said back panel, said straps having an interior and exterior surface and sized to extend from said first lateral side to a second opposite lateral side of said back panel and to substantially cover the Transverse Abdominis muscles of the body, said straps further comprising elastic and said garment further comprising releasable attachment elements located on said straps for attaching the ends of said straps to attachment members located on said second opposite lateral side.

2. The compression garment recited in claim 1 further comprising a front panel section wherein said front panel attaches said first lateral side of said rear panel to said second lateral side of said rear panel.

3. The compression garment of claim 2 wherein said front panel section comprise elastic material.

4. The compression garment of claim 2 wherein said front panel and said rear panel comprise elastic material.

5. The compression garment recited in claim 1 wherein said releasable attachment elements further comprise a hook and loop fastener located on the interior surface of said straps.

6. The compression garment recited in claim 5 wherein said attachment member on said second lateral side comprises a hook and loop fastening member that extends vertically from the top to the bottom said garment.

7. The compression garment recited in claim 4 further comprising hook and loop attachment elements located on the exterior surface of the said straps wherein the attachment member on the interior end surface of said strap is adapted to be attached to either said opposite side lateral attachment member provided on the lateral side of the garment or the exterior surface of one or more of the other said straps.

8. The compression garment recited in claim 1 further comprising shoulder straps said straps extending from the rear section to the front section.

9. The compression garment recited in claim 1 further comprising sleeves.

10. The compression garment recited in claim 1 further comprising a panty section that connects the front section to the rear section.

11. The compression garment recited in claim 10 wherein said panty section comprises a releasable fastener that allows the garment to take a tubular shape.

12. The compression garment recited in claim 5 further comprising hook and loop fasteners on the exterior surface of said straps wherein the straps may be overlapped wherein an attachment surface on the interior surface of a first strap may be attached to an exterior surface of a second strap.

13. The compression garment recited in claim 1 wherein the attachment means for said straps comprise buckles and said straps thread through said buckles wherein the tension on said straps can be adjusted by moving the strap through said buckle.

14. The compression garment recited in claim 1 wherein said attachment members comprise a plurality of snap members and snap receiving members that can be engaged at a plurality of locations to tighten and release tension provided by said straps.
